(19) United States
(12) Patent Application Publication
(10) Pub. No.: US 2013/0296824 A1
(43) Pub. Date: Nov. 7, 2013

(54) MICRONEEDLE BASED TRANSDERMAL DRUG DELIVERY DEVICE AND METHOD

(71) Applicants: Jianwei MO, Fremont, CA (US); Guanjun WANG, Fremont, CA (US)

(72) Inventors: Jianwei MO, Fremont, CA (US); Guanjun WANG, Fremont, CA (US)

(73) Assignee: UC BIODEVICES CORPORATION, Fremont, CA (US)

(21) Appl. No.: 13/463,464

(22) Filed: May 3, 2012

(51) Int. Cl. A61M 5/32

(52) U.S. Cl. 604/506; 604/173; 604/228

(57) ABSTRACT

A minimal pain microneedle based transdermal drug delivery device and method. The device has a clamshell configuration, where the top part of the clamshell holds one or more chambers configured to store liquid drugs, and also configured to store one or more spring operated plungers, and at least one microneedle. The top portion of the device is attached to the bottom portion of the device by a combination hinge and a moveable shutter mechanism. In its shut position, the shutter mechanism prevents the plungers from moving, and the open shutter position releases the plunger. When the user applies the bottom of the device to the user's skin and presses on the top portion with enough force to overcome a detent mechanism, the top portion pivots against the bottom portion forcing the microneedle through an aperture and into the skin painlessly. Pressing on the shutter mechanism then results in drug self-administration.

### BACKGROUND OF THE INVENTION

1. Field of the Invention

This invention is in the field of microneedle and hypodermic needle based transdermal drug delivery devices and methods.

2. Description of the Related Art

In recent years, there has been a high level of interest in the use of microneedle based devices for transdermal drug delivery. As discussed by McAllister et. al., “Microfabricated needles for transdermal delivery of macromolecules and nanoparticles: Fabrication methods and transport studies” Proceedings of the National Academy of Sciences, 100 (24), 13755-13760, (2003); and Wermeling et. al., Microneedles permit transdermal delivery of a skin-impermeant medication to humans, Proceedings of the National Academy of Sciences 105 (6), 2058-2063 (2008); micrometer scale needles can be useful for drug administration because their small size can potentially overcome many of the pain issues associated with traditional hypodermic needle approaches. This in turn can allow drugs, including drugs that might otherwise not be capable of being delivered by a conventional transdermal patch, to be delivered over longer periods of time in a transdermal patch like manner.

Such approaches are now feasible because recent advances in microfabrication technology now make it possible to fabricate such microneedles.

Further, the work of McAllister et. al. has provided clinical proof that micrometer scale needles (microneedles) can pierce through the outer stratum corneum barrier of human (and animal) skin, and deliver various useful drugs (such as Naltrexone) for prolonged periods of time (e.g. 48 hours or more).

As a result, there has been a substantial amount of interest in devising various types of microneedle based transdermal drug delivery devices.

For example, Yeshurun et. al., in U.S. patent publication 2008/0015522 taught a dual chamber injector integrated with micro-needles type device.

Fischer, in U.S. patent publication 2009/0234322 taught a method of dental tissue injection using an array of microneedles.

Cachemaille et. al., in U.S. patent publication 2010/02566569 taught a medical injection device with microneedles.

Tokumoto, et. al., in U.S. patent publication 2009/0030365 taught a transdermal drug administration apparatus having microneedles.

Beebe et. al., in U.S. patent publication 2011/0172601 taught at bladder arrangement for a microneedle-based drug delivery device.

Moga et. al., in U.S. patent publication 2011/0172609, taught a microneedle component assembly for a drug delivery device.

Prausnitz et. al., in U.S. Pat. Nos. 6,611,707 and 7,226,439 taught a microneedle drug delivery device.

Gonnelli et. al., in U.S. Pat. No. 8,070,726, taught a hydraulically actuated pump for long duration medicament administration that was capable of operating with microneedles.

Vedrine, in U.S. Pat. No. 7,857,131, taught a patch-like infusion device that could also operate with microneedles.

Yeshurun et. al., in U.S. Pat. No. 7,588,522, taught devices and methods for transporting fluid across a biological barrier that were also capable of operating with microneedles.

Gabel et. al., in U.S. Pat. No. 6,780,171, taught an intradermal delivery device that used microneedles.

Despite this prior teaching, microneedle based drug delivery devices are far from reaching their full potential and few if any such devices have been commercialized. Thus further advances in the field would be useful.

### BRIEF SUMMARY OF THE INVENTION

In one embodiment, the invention may be a microneedle based transdermal drug delivery device and method. The device may have a clamshell configuration, where the top part of the clamshell holds one or more chambers configured to store liquid drugs, and this top may also be configured to store one or more spring operated plungers, and at least one microneedle.

The top portion of the device may be attached to the bottom portion of the device by a unique combination hinge and moveable shutter mechanism. In its shut position, the shutter mechanism can prevent the spring operated plungers from moving, while in the open position, the shutter may release the plunger(s) from its locked position, thus allowing the one or more springs to force the one or more plungers into the one or more chambers containing one or more drugs. This force in turn pushes the drug(s) through the microneedle(s), and out into regions of the user's (recipient's) skin tissue below the stratum corneum.

When the user (device operator) applies the bottom of the device to the recipient's skin (either by pressing the device against the skin, or by attaching the device to the skin using an adhesive or other attachment mechanism) and presses on the top portion with enough force to overcome a detent mechanism, the top portion of the device pivots against the bottom portion of the device. This force pushes the device's one or more microneedles through an aperture in the bottom portion of the device, past the top stratum corneum layer of the skin, and into the lower layers of the epidermis or into the dermis or into the subcutaneous tissue (hypodermis). The user (operator) may then press on the shutter mechanism to move the shutter from closed to open, and this in turn will cause the plunger to move, forcing the drug out of the chamber, through the one or more microneedles, past the outer stratum corneum barrier layer of the skin, and into the skin's lower layers, dermis, or hypodermis. The device can thus inject a metered amount of drug, with minimal pain or discomfort for the user, for a short or potentially prolonged period of time.

### BRIEF DESCRIPTION OF THE DRAWINGS

FIG. 1A shows a 3-dimensional view of the device, looking down from the top.

FIG. 1B shows a partial cross sectional view of the device from the side.

FIG. 2A shows some interior details of the top portion of the device, showing the hinge opening and two hollow chambers, which may be used both to store drugs, and also to house the a spring driven plunger.

FIG. 2B shows some details of some various parts of the spring-driven plunger.

FIG. 3A shows a 3-dimensional view of the bottom portion of the device, again looking down from the top.

FIG. 3B shows a 3-dimensional view of the device's unique combination hinge and injection control shutter.

FIG. 3C shows a 3-dimensional view of the device, showing where the device's combination hinge and injection control shutter fits.

FIG. 4A shows a cross section of the device at the combination hinge and injection control shutter, showing the shutter in a “closed” configuration, thus locking the plungers into position and preventing them from pressing on the drug storage chambers.

FIG. 4B shows a cross section of the device at the combination hinge and injection control shutter, showing the shutter in an “open” configuration, where the plunger can now move in to force drug from the device's chamber(s) to the microneedle, and out to the user.

FIG. 5A shows a cross section of the device resting on a user's (recipient's) skin, while the clamshell device is still in an open configuration.

FIG. 5B shows a cross section of the device resting on a user's (recipient's) skin, while the clamshell device now in a closed configuration, the plunger is now pressing on the drug storage chamber, and drug is flowing through the microneedle and into the user's (recipient's) skin tissue.

FIG. 6A is a repeat of FIG. 5A, here shown to better illustrate the action of the spring driven plunger, shown in FIGS. 6B and 6C.

FIG. 6B shows the spring driven plunger before activation. Here drug is stored in the drug storage chamber, and in some embodiments, the base of the plunger may protrude out of an opening in the top portion of the device.

FIG. 6C shows the spring driven plunger after activation. The spring has returned to its normal (lowest energy state) more open coiled configuration, and in doing so has driven the plunger into the drug storage chamber, thereby forcing the drug out through the one or more microneedles (not shown).

FIG. 7A shows another cross section of the device at the combination hinge and injection control shutter, showing the shutter in an “open” configuration, where the plunger can now move in to force drug from the device's chamber(s) to the microneedle, and out to the user. Here the microneedle is protruding through the aperture, which in this configuration is bumped out somewhat from the lower surface of the device.

FIG. 7B shows a microneedle with five side openings, and a closed tip.

FIG. 7C shows a microneedle with five side openings and an open tip.

FIG. 7D shows a microneedle with three side openings and a closed tip

FIG. 7E shows a microneedle with no side openings and an open tip.

### DETAILED DESCRIPTION OF THE INVENTION

Note that although in a preferred embodiment, the device and method may be used by a human patient to self administer drugs, the device may also be used by another individual (e.g. healthcare professional, parent, other responsible individual) to administer drugs to a different person. Additionally the device and method may also be used to administer drugs to animals as well. Thus as used in this specification, the term “user” is intended to apply broadly to any drug recipient, living or non-living, human or non-human, regardless of if the recipient is actually self administering the drug or not. Because the system can often be used for drug self administration, for simplicity the term “user” will also refer to the operator of the device as well, however this simplified language is not intended to be limiting.

Similarly, terms such as “injection into the skin” and the like should be understood as meaning that the drug has been at least injected below the outer stratum corneum layer of the skin, and often into the recipient's dermis and hypodermis. In other words, the drug will be injected deeply enough to be absorbed into the body, not just reside on the surface of the user's (recipient's) skin.

In one embodiment, the invention may be a microneedle based transdermal drug delivery device, and a corresponding method for administering fluid drugs into the skin of a user.

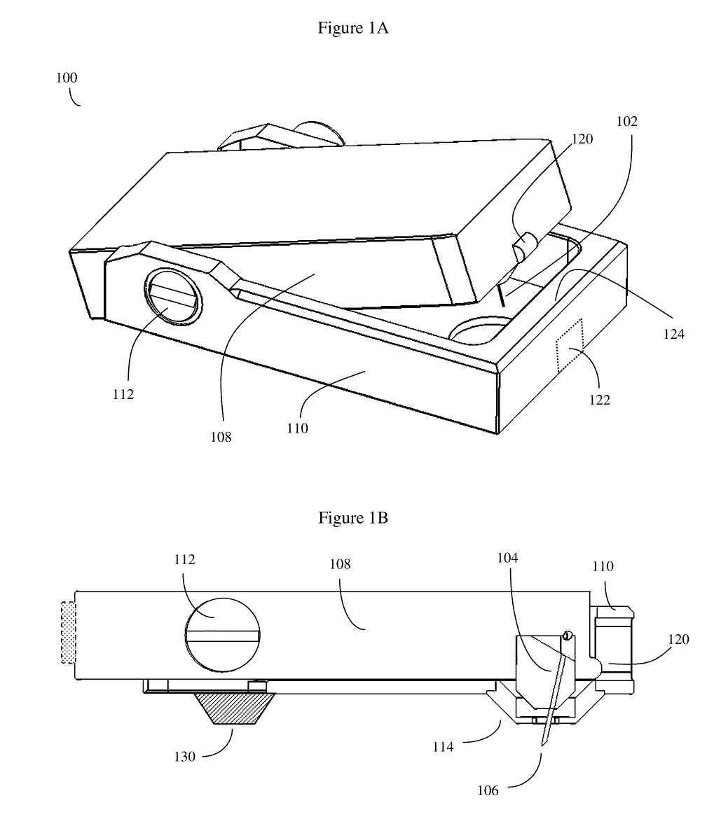

An overview of the device is shown in FIGS. 1A and 1B. FIG. 1A shows a 3-dimensional view of the device (100) in an open configuration, looking down from the top, and FIG. 1B shows a partial cut-away view of the device in the closed configuration, showing certain aspects of the interior of the device.

The device (100) will generally comprise at least one hollow interior microneedle (102), with a base end (104) and tip (106), mounted on the lower side of the top portion (108) of a hinged clamshell case. This clamshell case will in turn generally comprise a top portion (108), a bottom portion (110), and a hinge (112) by which the top portion (108) may pivot with respect to the bottom portion (110). This clamshell case is configured to administer drug to (and through) the skin of a user through the lower side (skin side) of the bottom portion of the clamshell case (114). This will be shown in more detail in subsequent figures.

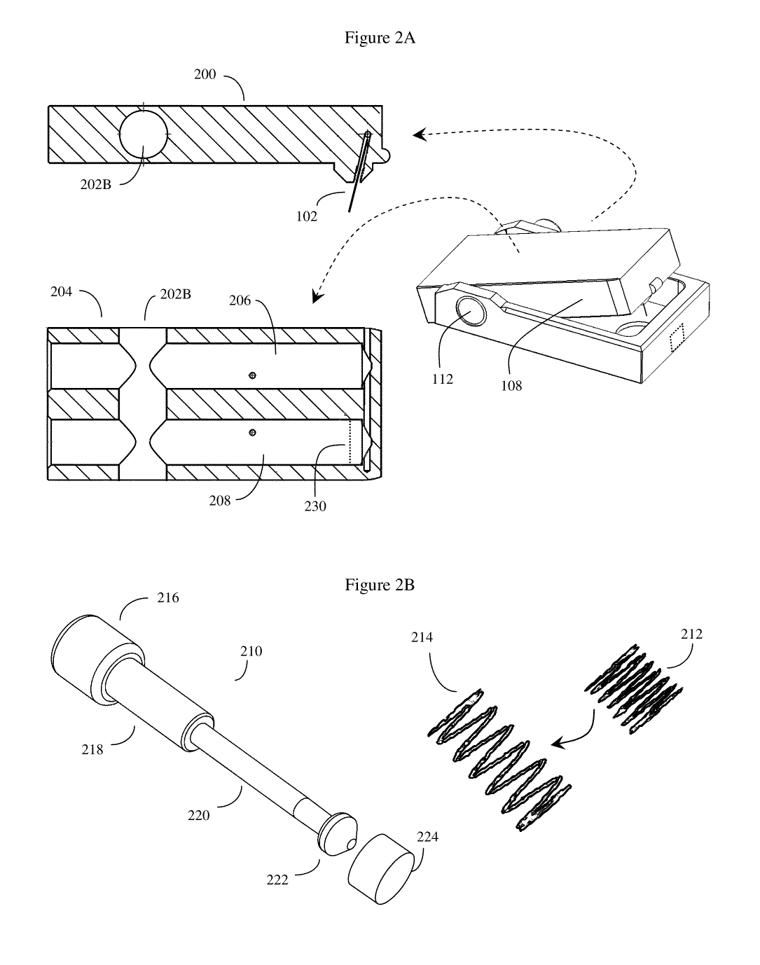

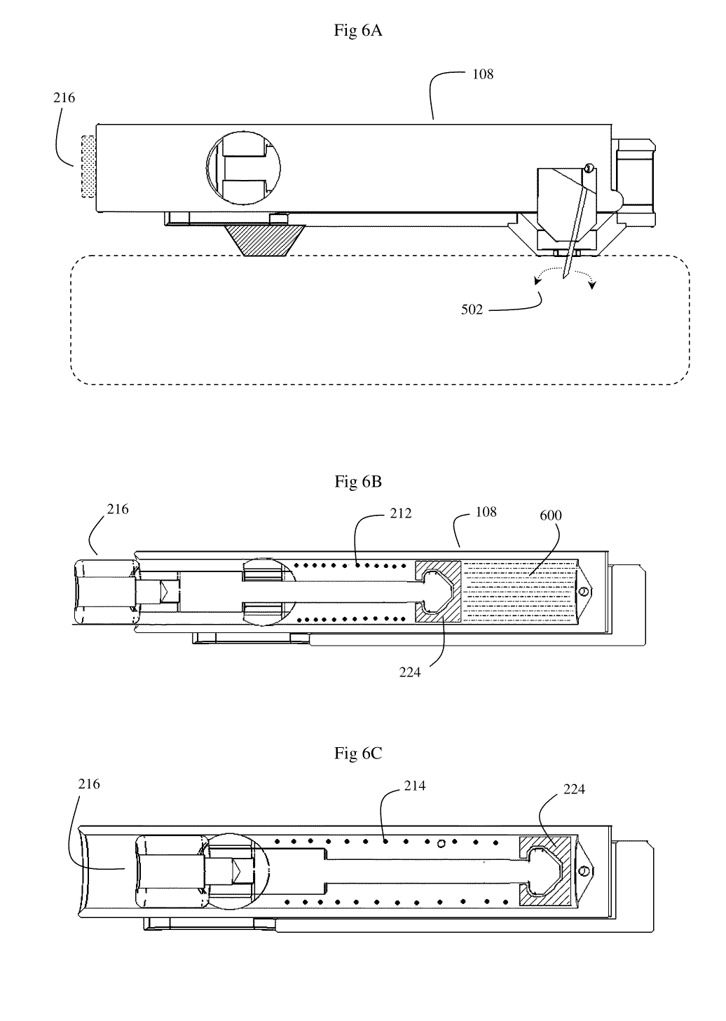

The top portion (108) of the hinged clamshell case additionally comprises at least one hollow chamber for storage of a fluid drug or a solid drug and a fluid. This is shown in more detail in FIGS. 2A, 6B, and 6C.

FIG. 2A shows the top portion of the hinged clamshell case (108) from two different, cross section viewpoints. FIG. 2A (200) shows a cross section side view of this top portion (108), here focusing on the hole (202) for the hinge (112), and the microneedle (102). FIG. 2A (204) shows a top view cross section (204) of the top portion (108) of the device. As can be seen from this cross section view (204), in this particular embodiment, there are two interior hollow chambers (206), (208) which can be filled with one or more drugs (either solid, or fluid (e.g. liquid, liquid suspension)). The chambers can also hold a spring and plunger mechanism to deliver these one or more drugs.

In some embodiments, such as the embodiments shown FIG. 2A, these at least one hollow chamber(s) (e.g. 206, 208) may be disposed approximately parallel to the lower side of the top portion (108) of the hinged clamshell case.

The one or more hollow chambers (206), (208) may be configured for each chamber to accept a spring-operated plunger. This plunger is shown in FIG. 2B (210), and the spring in both a normal (e.g. extended 214) and compressed (e.g. more tightly coiled 212) configuration is also shown. This plunger (210) will often have a head end (216) which may be designed to protrude outside of the top portion (108) of the case before the drug is administered. The plunger may also have a larger diameter middle portion (218), a smaller diameter middle portion (220), and a first tip (222) which in turn will often be affixed to or make contact with a deformable synthetic rubber or other semi-rigid material plunger tip (224). The plunger tip will (224) often make direct contact with the drug that is stored in the hollow chamber(s) (206), (208), so generally the plunger tip materials will be chosen to both prevent leakage of the drug, and also not to adversely chemically interact with the drug.

The spring (214), (212) will generally wind around the smaller diameter middle portion (220) of the tip (210), (see FIGS. 6B and 6C) and will generally be configured to, in its lowest energy state, preferentially assume its normal, more extended configuration (214) (FIG. 6C). The spring (212), (214) and plunger (210), once in place in a hollow chamber (206), (208) thus form a spring operated plunger (FIG. 6B). The tendency of the spring to expand to its normal shape (214) (FIG. 6C) will provide a force to move the plunger inward.

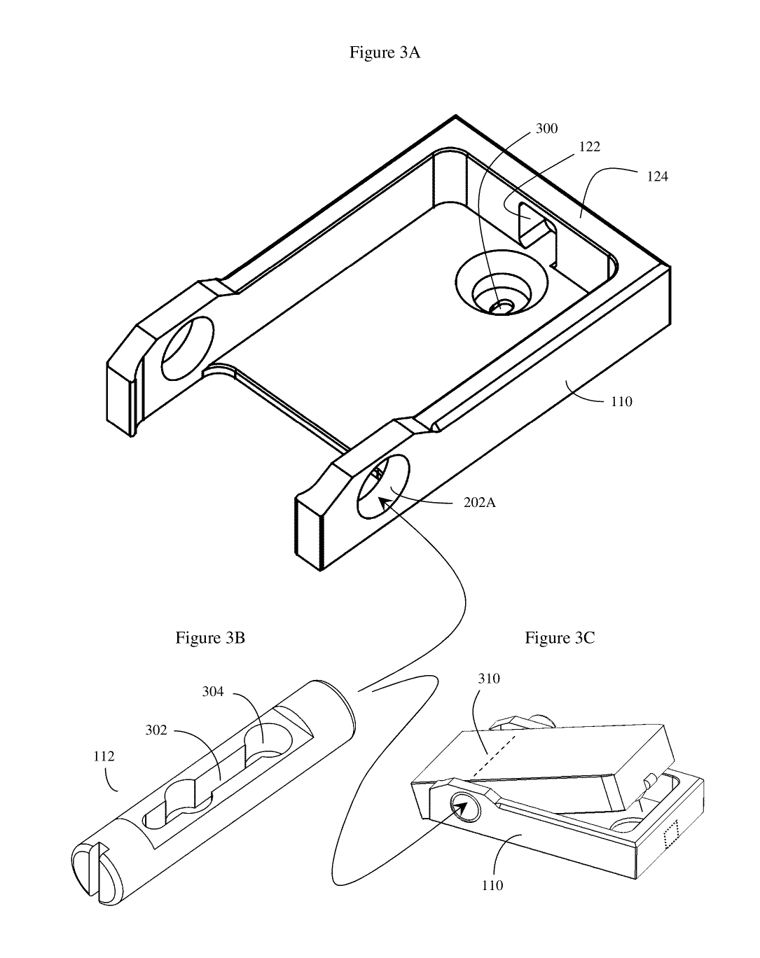

The spring operated plunger is normally prevented from pushing into the hollow chamber (206), (208) by the action of a moveable combination hinge and shutter (112) disposed to alter configurations between a first position that mechanically blocks movement of the spring operated plunger, and a second position that allows movement of the spring operated plunger. This moveable shutter (112), which in some embodiments may also function as the device hinge, is shown in FIG. 3B.

FIG. 3A shows a 3-dimensional view of the bottom portion of the device (110), showing both the hole for the hinge (202), and other elements to be discussed shortly. Here the combination hinge and shutter (112) fits through hinge holes (202A), (202B) in both the bottom portion (110) and top portion (108) of the device.

The shutter (112) will often have slots and holes (302), (304), where the slots are designed to accommodate the narrower diameter middle portion of the plunger (220), while the holes are designed to accommodate the larger diameter middle portion of the plunger (218).

The bottom portion (110) of the hinged clamshell case is also configured with at least one aperture (300) disposed to allow the at least one microneedle (102), when the top portion (108) of the clamshell case rotates about the hinge (112) and makes contact with the bottom portion (110) of the clamshell case, to penetrate through the aperture (300) (e.g. a hole in the bottom of (110) and into the skin of a device user (e.g. the recipient of the drug). In some configurations, the aperture (300) is flush with the bottom of the lower portion (110), while in other configurations, the bottom portion (110) may be configured with one or more protruding bumps, and the aperture (300) can be a hole in one or more of these one or more protruding bumps.

Note that although FIG. 3A (300) shows only a single oval shaped aperture designed to accommodate only a single microneedle, in alternative embodiments, the device may comprise a plurality of microneedles (e.g. a microneedle array patch). In this case, aperture (300) could be an alternative opening shape, such as a circle, oval, square, rectangular, cross, or star type opening.

Also as shown in FIG. 3A (300) and also in other Figures such as 1B, and 7A, although the aperture may be flush with the bottom portion of the device (110), often the aperture may further protrude out from the surface of the bottom portion of the hinged clamshell case, thereby forming a bumper.

The device may contain other bumpers as well, which may not contain apertures, but which rather may be intended to keep the device relatively parallel with the user's (recipient's) skin. Such additional bumpers are optional. An example of such an optional additional bumper is shown in FIG. 1B (130), 5A (130), and elsewhere.

Alternatively, the underside of the device may contain an adhesive to help adhere the device to the user's (recipient's) skin for longer periods of time. In this case, (130) may be regarded as a section of adhesive, which in fact may extend to cover a substantial amount (e.g. 50% or more) of the underside of the device.

Often the top portion (108) of the hinged clamshell case and the bottom portion of the clamshell case (110) will also be configured with at least one detent structure. This detent structure may be configured to prevent the top portion (108) of the clamshell case and the bottom portion of the clamshell case (110) from completely shutting unless the user (e.g. operator—here anyone operating the device) presses with sufficient force on the top (108) of the clamshell case as to force the top (108) of the clamshell case to overcome the resisting force of the detent, pivot with respect to the hinge (112), and press against the bottom portion of the clamshell case (110).

This detent structure may be formed in many different ways. As one example, the top portion of the device may contain a slightly protruding portion (120) that acts like a deformable lever, and the bottom portion of the device may contain an optional notch or hole (122) designed to capture this protruding portion (120). The protruding portion of the detent (120) normally will not pass through the barrier imposed by a flange (124) around the bottom portion (110) of the device, but when pressure is applied, the protruding portion (120) will deform enough to pass through, and then it will be captured by hole or notch (122). The detent structure may have one or multiple protruding portions, and one or respectively positioned multiple notches or holes, respectively. The protruding portions may be on the top or bottom portion of the clamshell device, while the corresponding notches may be on the bottom or top portion of the clamshell device.

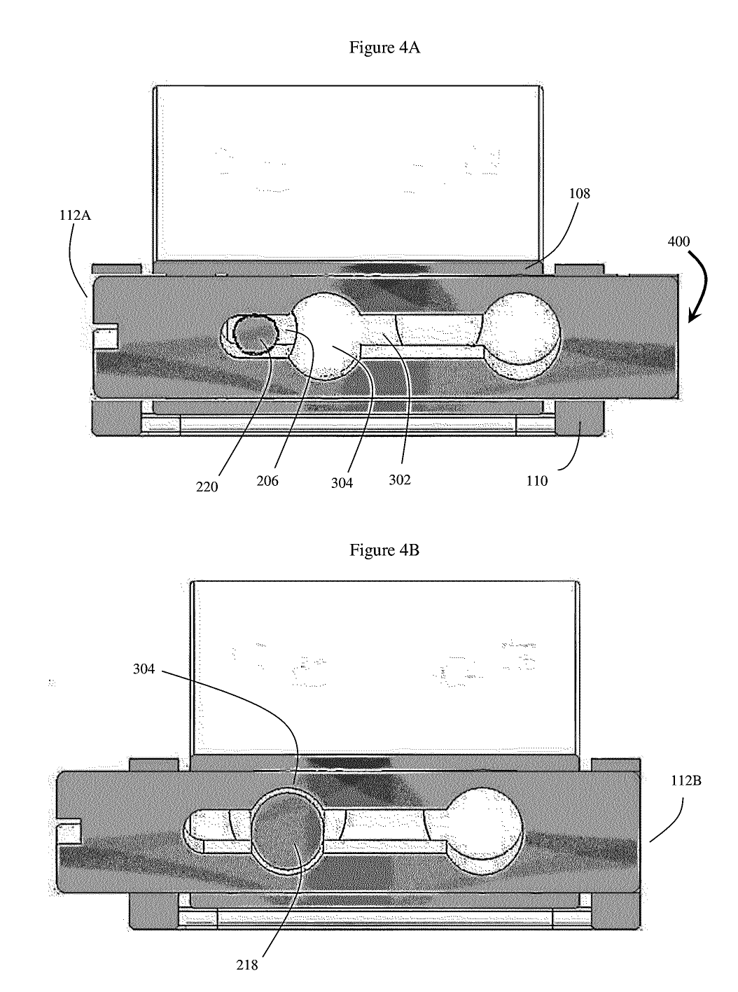

FIGS. 4A and 4B shows more details of how shutter (112) may operate to control the plunger (210) and thus drug administration. FIGS. 4A and 4B show a cross section of the device as if cut through the top, bottom, and hinge region of the device as shown in FIG. 3C (310).

In FIG. 4A, the shutter is in a first “shut” configuration (112A). In this position, slot (302) of the shutter (112) is positioned so that, when plunger (210) is positioned in hollow chamber (206), only the smaller diameter middle portion (220) of plunger (210) can pass through the slot (302). The larger diameter middle portion (218) of the plunger is blocked, and thus the plunger cannot, even though driven by force from spring (214), go further into the hollow chamber (206).

The user (operator) can move the shutter from the first “shut” configuration to the second “open” position by, for example, pressing (applying force) (400) to the end or side of the shutter. Note that this shutter opening step (400) will usually occur after the clamshell closing step (502) has been done.

In FIG. 4B, the shutter (112) is now in a second “open” position (112B). As previously discussed, the shutter can be opened by, for example, finger pressure (400) against the sides of the shutter as they protrude out of the openings (202A). In its second “open” position (112B), the holes in the shutter (304) are now positioned so as to accommodate the larger diameter middle portion (218) of plunger (210). As a result, urged on by the force of the compressed spring (212) (FIG. 6B) attempting to regain its normal extended coiled shape (214) (FIG. 6C), the plunger is now free to allow spring (212), (214) to force the plunger further towards the back of chamber (206).

The device is generally configured so that at least one chamber (206), (208) is filled with a fluid drug. Alternatively, in some multiple chamber embodiments, one chamber may contain a solid, highly viscous drug, or first form of a drug, and the other chamber may contain a drug diluent or other drug activator. The device is also configured so that when the user places the lower (skin side) bottom portion (110) of the device against the user's skin (e.g. the recipient's skin), and presses on the upper side of the top portion (108) of the clamshell device, at least one microneedle (102) penetrates through the at least one aperture (300) and into the skin.

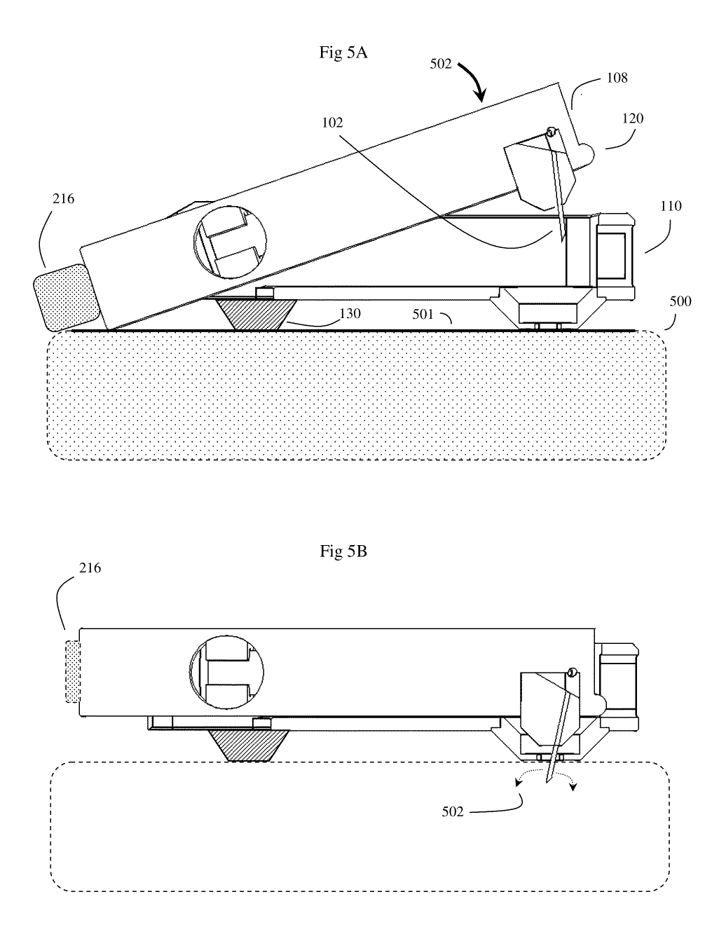

These steps are shown in more detail in FIGS. 5A, 5B, 6A, 6B, and 6C.

In FIG. 5A, the device (100) is shown pressed against the user (e.g. recipient's) skin (500). The details of the inner layers of the skin, as well as portions of the device, are shown in cross section, and the hinge/shutter (112) is not shown, to better convey what is happening. Here the stratum corneum layer of the skin is shown as the solid line (501), while, the various layers below the stratum corneum are shown as either dotted regions or, to better visualize drug flow, as white regions. After the user (e.g. the device operator) presses the device against the user's (e.g. recipient's) skin, the user/device operator applies force (502) to the upper side of the top portion of the device.

Force (502) causes the top portion of the device (108) to rotate about the hinge/shutter (112—not drawn). It also forces the protruding portion of the detent (120) past the barrier imposed by the flange (124) around the bottom portion (110) of the device and into the hole or notch (122). The needle (102) is also forced through the aperture (300), past the stratum corneum, and into the lower layers of the user's (recipient's) skin.

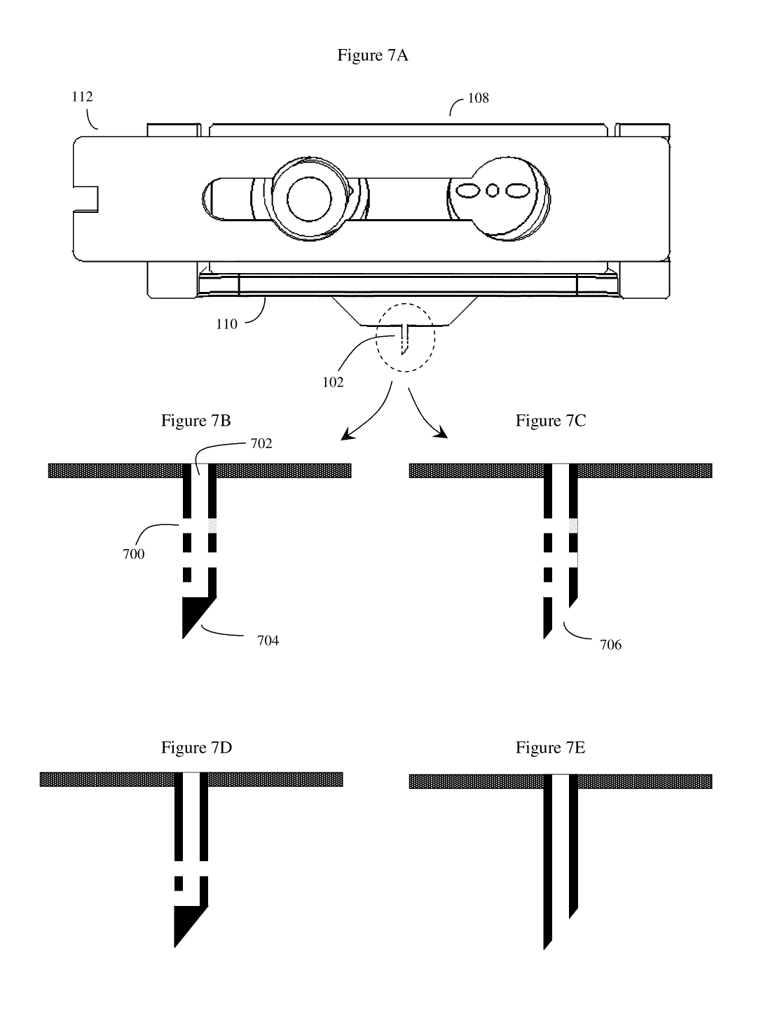

After the user (operator) then opens the shutter (112A to 112B, see FIGS. 4A and 4B), the one or more plungers (210) can then move further into the one or more chambers (e.g. 206, 208). This will be shown in more detail in FIGS. 6A, 6B, and 6C. The net effect is that the plunger (210) moves further into the chamber, forcing the drug in the chambers out into the needle (102), past the outer layer of the user's (recipient's) skin, through the needle holes (see FIG. 7) and into (504) the inner layers (e.g. the transdermal region) of the skin.

FIGS. 6A, 6B, and 6C show how the spring operated plunger works in more detail. FIG. 6A shows a cross section of the device injecting drugs into the user's skin, while FIGS. 6B and 6C show more details of the plunger in operation.

In FIG. 6B, a cross section of the top portion (108) of the device is shown before the shutter (112) has been opened. At this point, the chamber(s) (206), (208) are filled with drug (600), which will often be a fluid drug (e.g. a liquid or a liquid suspension). Alternatively, as previously discussed, one chamber (e.g. 206) could be filled with a liquid, while a different chamber (e.g. 208) could be filled with a solid drug that is dissolved by the liquid (e.g. diluent) as the plunger(s) advance. Here any permutation of solid, liquid, or suspension drugs, multiple drugs, diluents, drug precursors and activating agents, and the like is contemplated and may be used in this device.

In FIG. 6B, the shutter has now been opened, and the plunger (210) has advanced to the end of the chamber (206), (208), and essentially all of the drug (600) has now been expelled through the needle. Note that the spring changed from its original compressed configuration (212) in FIG. 6B to its more relaxed (lower energy state) open configuration (214) in FIG. 6C, and in this embodiment, this spring provides the driving force to both move the plunger, and force the drug (600) through the microneedle (102).

Thus, as previously discussed, when the operator further moves the moveable shutter (112), (400); the at least one spring operated plunger (210) pushes into the at least one chamber (206), (208), forcing the fluid drug (600) into the hollow interior of the microneedle (102) and into the skin (502) of the user/recipient.

Various types of microneedles may be used with this device, and some of these various microneedle configurations are shown in FIGS. 7A-7E.

FIG. 7A shows another view of the device, looking forward from the perspective of FIG. 3C, line (310). In particular, this view shows microneedle (102) protruding out from the aperture (300) when the device is shut and the top portion of the device (108) has rotated on the hinge/shutter (112) and is now pressed against the bottom portion of the device (110).

The device's one or more microneedles may often each generally comprise least one side opening communicating with the hollow interior of the microneedle. In FIG. 7B, these side openings are shown as (700), and the hollow interior of the microneedle is shown as (702). As shown in FIGS. 7B and 7D, in some embodiments, the tip (704) of the microneedle may be closed. This can help prevent clogging the hollow interior (700) with skin debris when the microneedle is inserted into the skin. In other embodiments, the tip of the microneedle may be open (706). The microneedle is fed with the fluid drug coming from the hollow chamber(s) such as (206) and (208).

Generally, the microneedle (102) will have a length of greater than about 300 microns, and a diameter of less than about 1200 microns. Thus the term “microneedle” can, in fact, encompass both micron-scale microneedles, on up in size to standard fine-gauge hypodermic needles. These microneedle (or fine-gauge hypodermic needles) can be either straight or curved.

In some embodiments, at least one of the one or more hollow chambers (see FIG. 2A, 230) may be prevented from being in fluid communication with the hollow interior of the one or more microneedles (702) by a foil or membrane FIG. 2A (300). This foil or membrane may be ruptured when either the user (operator) presses (502) against the top portion (108) of the clamshell device, or when the user (operator) moves the movable shutter (e.g. FIGS. 4A, 4B 112A and 112B), or when the user (operator) pulls some other type of release tab or device. This foil or membrane can help prevent drug evaporation, or clogging of the microneedle bore by evaporated drug residue.

### Alternative Embodiments

In some embodiments, it may be desirable to have the microneedle (102) pass through the aperture (300) and through the user's (recipient's) stratum corneum with a standardized amount of force. In these embodiments, the top portion, bottom portion, and hinge of the hinged clamshell case may be further configured with a shutting spring so that when the user (operator) presses with sufficient force (502) on the top (108) of the device, to cause the top (108) to overcome the resisting force of the detent (120), (122), (124) a clamshell shutting spring or other mechanism then takes over to force the top portion (108) down against the bottom portion (110) with a standardized amount of force.

In some embodiments, it may be desirable to hold the spring force with one or multiple switchable stoppers or shutters (instead of one slidable shutter) to prevent the plunger(s) from pushing into the chambers. When one of the switchable stoppers or shutters is released (removed), the compressed spring (similar to (212) in FIG. 6B) will regain its normal extended coiled shape (similar to (214) in FIG. 6C), the plunger is now free to allow spring (212), (214) to force the plunger further towards the back of chamber (206). This plunger then pushes the stored drugs in the chamber out into the user's skin through either a single or multiple microneedles. These embodiments can permit multiple doses of drugs to be intermittently delivered into the user's skin tissue, on demand, at the desirable time.

Note that although in a preferred embodiment, the device will function with at least one, and often a plurality of microneedles (e.g. a patch of microneedles), use with microneedles is not always strictly necessary. In other embodiments, standard fine gauge hypodermic needles, such as 18, 21, 26, 29 or 33 gauge needles, and the like, may also be used.

In some embodiments, it may be useful to provide the device with an adhesive underlayer capable of attaching the underside of the device to the skin for a prolonged period of time, such as one or more days. In such embodiments, it may additionally be useful to configure the force of the spring, the diameter of the various fluid conducting conduits and openings, and the viscosity of the fluid drug in such a way that the device may continuously deliver drug in a relatively uniform manner over this prolonged period of time (e.g. on the order of a day or more).

1. A microneedle based transdermal drug delivery device, for administering fluid drugs into the skin of a user, comprising:
at least one hollow interior microneedle, with a base end and tip, mounted on the lower side of the top portion of a hinged clamshell case;
said clamshell case comprising a top portion, a bottom portion, and a hinge by which said top portion may pivot with respect to said bottom portion, said clamshell case configured to administer drug to said skin of said user through the lower skin side of said bottom portion of said clamshell case;
said top portion of said hinged clamshell case additionally comprising at least one hollow chamber for storage of a fluid drug or a solid drug and a fluid;
each said at least one hollow chamber configured to accept a spring operated plunger;
said spring operated plunger being prevented from pushing into said hollow chamber by the action of a moveable shutter disposed to mechanically block movement of said spring operated plunger;
said bottom portion of said hinged clamshell case configured with at least one aperture disposed to allow said microneedle, when said top portion of said clamshell case makes contact with said bottom portion of said clamshell case, to penetrate through said aperture and into said skin of said user;
said top portion of said hinged clamshell case and said bottom portion of said clamshell case configured with at least one detent structure configured to prevent said top portion of said clamshell case and said bottom portion of said clamshell case from completely shutting unless said user presses with sufficient force on said top of said clamshell case as to force said top of said clamshell case to overcome the resisting force of said detent, pivot with respect to said hinge, and press against said bottom portion of said clamshell case;
said device configured so that when said at least one chamber is filled with a fluid drug, and said user places the lower skin side of said bottom portion of said clamshell device against said skin, presses on the upper side of the top portion of said clamshell device, said at least one microneedle penetrates through said at least one aperture and into said skin; and
when said user further moves said moveable shutter; said at least one spring operated plunger pushes into said at least one chamber, forcing said fluid drug into said hollow interior of said microneedle and into the skin of said user.

2. The device of claim 1, wherein each said at least one microneedle has at least one side opening communicating with said hollow interior of said microneedle.

3. The device of claim 2, wherein each said at least one microneedle additionally has a closed tip.

4. The device of claim 1, wherein said at least one hollow chamber is in fluid communication with the hollow interior of said at least one hollow microneedle.

5. The device of claim 1, wherein at least one of said at least one hollow chamber is prevented from being in fluid communication with the hollow interior of said at least one hollow microneedle by a foil or membrane that is ruptured when either said user presses against said top portion of said clamshell device, or when said user moves said movable shutter, or when said user pulls a release tab.

6. The device of claim 1, wherein said at least one hollow chamber is disposed approximately parallel to the lower side of said top portion of said hinged clamshell case.

7. The device of claim 1, wherein said at least one microneedle has a length of greater than 300 microns and a diameter of less than 1200 microns; and wherein said at least one microneedle is straight or curved.

8. The device of claim 1, wherein said aperture comprises a circle, oval, square, rectangular, cross, or star type opening.

9. The device of claim 1, wherein said top portion, bottom portion, and hinge of said hinged clamshell case are further configured with a shutting spring so that when said user presses with sufficient force on said top of said clamshell case as to force said top of said clamshell case to overcome the resisting force of said detent, said shutting spring forces said top portion against said bottom portion with a standardized force.

10. The device of claim 1, wherein said aperture further protrudes out from the surface of said bottom portion of said hinged clamshell case, thereby forming a bumper.

11. A microneedle based transdermal drug delivery device, for administering fluid drugs into the skin of a user, comprising:
at least one hollow interior microneedle, with a base end and tip, mounted on the lower side of the top portion of a hinged clamshell case;
said at least one microneedle having at least one side opening communicating with said hollow interior of said microneedle;
said clamshell case comprising a top portion, a bottom portion, and a hinge by which said top portion may pivot with respect to said bottom portion, said clamshell case configured to administer drug to said skin of said user through the lower skin side of said bottom portion of said clamshell case;
said top portion of said hinged clamshell case additionally comprising at least one hollow chamber for storage of a fluid drug or a solid drug and a fluid;
said at least one hollow chamber being disposed approximately parallel to the lower side of said top portion of said hinged clamshell case;
each said at least one hollow chamber configured to accept a spring operated plunger;
said spring operated plunger being prevented from pushing into said hollow chamber by the action of a moveable shutter disposed to mechanically block movement of said spring operated plunger;
said bottom portion of said hinged clamshell case configured with at least one aperture disposed to allow said microneedle, when said top portion of said clamshell case makes contact with said bottom portion of said clamshell case, to penetrate through said aperture and into said skin of said user;
said top portion of said hinged clamshell case and said bottom portion of said clamshell case configured with at least one detent structure configured to prevent said top portion of said clamshell case and said bottom portion of said clamshell case from completely shutting unless said user presses with sufficient force on said top of said clamshell case as to force said top of said clamshell case to overcome the resisting force of said detent, pivot with respect to said hinge, and press against said bottom portion of said clamshell case;
said device configured so that when said at least one chamber is filled with a fluid drug, and said user places the lower skin side of said bottom portion of said clamshell device against said skin, presses on the upper side of the top portion of said clamshell device, said at least one microneedle penetrates through said at least one aperture and into said skin; and
when said user further moves said moveable shutter; said at least one spring operated plunger pushes into said at least one chamber, forcing said fluid drug into said hollow interior of said microneedle and into the skin of said patient.

12. The device of claim 11, wherein each said at least one microneedle additionally has a closed tip.

13. The device of claim 11, wherein said at least one hollow chamber in fluid communication with the hollow interior of said at least one hollow microneedle; or
wherein at least one of said at least one hollow chamber is prevented from being in fluid communication with the hollow interior of said at least one hollow microneedle by a foil or membrane that is ruptured when either said user presses against said top portion of said clamshell device, or when said user moves said movable shutter, or when said user pulls a release tab.

14. The device of claim 11, wherein said at least one microneedle has a length of greater than 300 microns and a diameter less than 1200 microns; and wherein said at least one microneedle is straight or curved.

15. The device of claim 11, wherein said top portion, bottom portion, and hinge of said hinged clamshell case are further configured with a shutting spring so that when said user presses with sufficient force on said top of said clamshell case as to force said top of said clamshell case to overcome the resisting force of said detent, said shutting spring then forces said top portion against said bottom portion with a standardized force.

16. A method of administering fluid drugs into the skin of a user, comprising:
obtaining a microneedle based transdermal drug delivery device,
said device comprising at least one hollow interior microneedle, with a base end and tip, mounted on the lower side of the top portion of a hinged clamshell case;
said clamshell case comprising a top portion, a bottom portion, and a hinge by which said top portion may pivot with respect to said bottom portion, said clamshell case configured to administer drug to said skin of said user through the lower skin side of said bottom portion of said clamshell case;
said top portion of said hinged clamshell case additionally comprising at least one hollow chamber for storage of a fluid drug or a solid drug and a fluid;
each said at least one hollow chamber configured to accept a spring operated plunger;
said spring operated plunger being prevented from pushing into said hollow chamber by the action of a moveable shutter disposed to mechanically block movement of said spring operated plunger;
said bottom portion of said hinged clamshell case configured with at least one aperture disposed to allow said microneedle, when said top portion of said clamshell case makes contact with said bottom portion of said clamshell case, to penetrate through said aperture and into said skin of said user;
said top portion of said hinged clamshell case and said bottom portion of said clamshell case configured with at least one detent structure configured to prevent said top portion of said clamshell case and said bottom portion of said clamshell case from completely shutting unless said user presses with sufficient force on said top portion of said clamshell case as to force said top portion of said clamshell case to overcome the resisting force of said detent, pivot with respect to said hinge, and press against said bottom portion of said clamshell case;
filling said at least one chamber with at least one fluid drug;
placing the lower skin side of said bottom portion of said clamshell device against said skin;
pressing on the upper side of the top portion of said clamshell device, thereby overcoming the resisting force of said at least one detent, and causing said at least one microneedle to penetrate through at least one aperture and into said skin;
moving said moveable shutter, thereby enabling said at least one spring operated plunger to push into said at least one chamber, forcing said fluid drug into said hollow interior of said microneedle and into the skin of said user.

17. The method of claim 16, wherein said at least one hollow chamber is in fluid communication with the hollow interior of said at least one hollow microneedle; or
wherein at least one of said at least one hollow chamber is prevented from being in fluid communication with the hollow interior of said at least one hollow microneedle by a foil or membrane; further removing or rupturing said foil or membrane during said method, thereby placing said at least one hollow chamber in fluid communication with the hollow interior of said at least one hollow microneedle.

18. The method of claim 16, wherein each said at least one microneedle has at least one side opening communicating with said hollow interior of said microneedle.

19. The method of claim 18, wherein each said at least one microneedle additionally has a closed tip.

20. The method of claim 16, wherein said at least one microneedle has a length of greater than 300 microns and a diameter less than 1200 microns; and wherein said at least one microneedle is straight or curved.

21. The method of claim 16, wherein said top portion, bottom portion, and hinge of said hinged clamshell case are further configured with a shutting spring so that when said user presses with sufficient force on said top of said clamshell case as to force said top of said clamshell case to overcome the resisting force of said detent, said shutting spring then forces said top portion against said bottom portion with a standardized force.
